(19) United States
(12) Patent Application Publication
(10) Pub. No.: US 2007/0191560 A1
(43) Pub. Date: Aug. 16, 2007

(54) Norbornenylmethyl fluoroalkyl ethers

(76) Inventors: Brian Edward Kipp, Avondale, PA (US); Lee G. Sprague, Augusta, GA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/698,488

(22) Filed: Jan. 25, 2007

Related U.S. Application Data
(60) Provisional application No. 60/772,622, filed on Feb. 13, 2006.

(51) Int. Cl. C08F 16/24; C07C 43/18

(52) U.S. Cl. 526/247; 568/670

(57) ABSTRACT

New monomers, norbornenylmethyl fluoroalkyl ethers, made by reaction of bicyclo(2,2,1)hept-5-ene-2-methanol with fluoro(alkyl vinyl ethers), are described. These monomers are useful in making polymers such as hydrophobic, hydrolytically-resistant polymers for photoresists, particularly for use in photoimaging by immersion lithography with 193 nm light.

### FIELD OF THE INVENTION

The present invention relates to novel monomers made by reacting bicyclo(2.2.1)hept-5-ene-2-methanol (CAS No. 95-12-5), referred to herein as 5-norbornene-2-methanol, with fluoro(alkyl vinyl ethers), the monomers being useful in making polymers for photoresists.

### BACKGROUND OF THE INVENTION

In photoimaging for making integrated circuits, photoresists are exposed to a specific wavelength of light through a photomask, which causes a change in solubility of the photoresist. A common mechanism for this change is the reaction of a photoacid generator with a photon, which generates acid, which in turn reacts with the polymer of the photoresist, changing its solubility. This change in solubility allows for selective removal of the photoresist to enable etching of the silicon substrate in the regions where the photoresist is selectively removed. Photoresists must be mostly transparent to the wavelength of light used, and are therefore the polymer of the photoresist is typically amorphous, that is, non-crystalline, exhibiting no melting endotherm when subjected to differential scanning calorimetry (DSC). Photoresists also have a mechanism for modifying solubility upon exposure to light, and preferably have a high glass transition temperature (Tg). Cyclic structures, such as norbornene, contribute to high Tg by virtue of their ring structure, but do not introduce functionality that affects the absorption of light, in particular at 193 nm.

The ability of photoresists to be used in making increasingly fine structures has been extended using a technique called immersion lithography. Immersion fluids with a targeted refractive index are able to focus the wavelength of light to create smaller (finer) features. A key immersion fluid is water which has added additional requirements to the photoresist, hydrophobicity and resistance to hydrolysis. In immersion lithography, the photoresist must not be swollen or dissolved by the immersion fluid.

Fluorocarbon substituents on the norbornenyl monomer contribute hydrophobicity. As stated, it is desirable that such substituents not be susceptible to loss through hydrolytic attack on the polymer. U.S. Pat. No. 4,751,168 discloses norbornenyl monomers made by Diels-Alder addition of cyclopentadiene and partially fluorinated vinyl compounds, vinyl esters, and vinyl ethers.

There is a need for additional partially fluorinated norbornene monomers that can be easily synthesized from available starting materials.

### SUMMARY OF THE INVENTION

In one embodiment this invention relates to R—CH₂—O—C₂FₐH_{b}—O—CₓF_{y}H_{z}Oₙ where R is bicyclo[2.2.1]hept-2-enyl and is bonded to the methylene group at the 5-position; a=1 to 3 and b=1 to 3, a+b=4; x=1 to 10, y=0 to 2x+1, z=0 to 2x+1, y+z=2x−1 or 2x+1;and n=0 to 8 with the proviso that the oxygen is ether oxygen. In a second embodiment this invention relates to polymerized R—CH₂—O—C₂FₐH_{b}—O—CₓF_{y}H_{z}Oₙ where R is bicyclo[2.2.1]hept-2-enyl and is bonded to the methylene group at the 5-position; a=1 to 3 and b=1 to 3, a+b=4; x=1 to 10, y=0 to 2x+1, z=0 to 2x+1, y+z=2x−1 or 2x+1; and n=0 to 8 with the proviso that the oxygen is ether oxygen.

### DETAILED DESCRIPTION OF THE INVENTION

The norbornenylmethyl fluoroalkyl ethers of the present invention are made by reacting norbornenyl methanol, specifically 5-norbornene-2-methanol (I), and fluoro(alkyl vinyl ether). The hydroxyl group of (I) adds 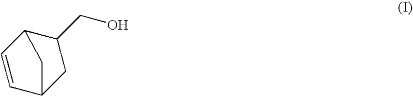
across the vinyl group of the fluoro(alkyl vinyl ether). The reaction may be represented as (Norb-OH being the alcohol (I), and CF₂═CF—OR being a fluoro(alkyl vinyl ether)):
Norb-OH+CF₂═CF—OR→Norb-O—CF₂—CHF—OR
As shown, the reaction product may be referred to herein as “adduct”. The reaction is base-catalyzed and proceeds under mild conditions. The reaction is exothermic and is preferably carried out at room temperature (about 25° C.) or below. A preferred temperature range is 20-25° C. when perfluoro(propyl vinyl ether) (PPVE) (boiling point 35-36° C.) is the vinyl ether. Perfluoro(ethyl vinyl ether) (PEVE) is lower boiling (7° C.) but the preferred temperature range is still satisfactory. Perfluoro(methyl vinyl ether) (PMVE) is a gas at room temperature (boiling point −22° C.) and this is best reacted in an autoclave. However, it is still desirable to keep the temperature below 25° C., to prevent undesirable secondary reactions such as the dehydrofluorination of the adduct by caustic to generate a vinyl ether. The undesirable dehydrofluorination reaction is:
Norb-O—CF₂—CFH—O—R+KOH→Norb-O—CF═CF—O—R+KF+H₂O
By keeping the reaction temperature below 25° C., dehydrofluorination is effectively suppressed.

Cooling of the reaction vessel with cold water or ice water is beneficial for maintaining reaction temperature. Temperature may also, or additionally, be controlled by adding the fluoro(alkyl vinyl ether) to the 5-norbornene-2-methanol at a rate such that the reaction temperature can be maintained at the desired point. It is further preferable to keep air (oxygen) away from the fluoro(alkyl vinyl ether) as these ethers react with oxygen. Sweeping and then blanketing the reaction vessel with inert gas, such a nitrogen, will sufficiently prevent oxygen from reacting with the vinyl ether. It is preferred that an aprotic polar solvent be used. Dimethyl sulfoxide (DMSO) is such a preferred solvent.

The preferred reaction procedure is to charge the reaction vessel with DMSO and aqueous caustic, preferably aqueous potassium hydroxide (at least about 25% KOH by weight, preferably at least about 35%, and more preferably at least about 40%, preferably no more than about 55%, more preferably no more than about 50%, and most preferably, about 45%), and 5-norbornene-2-methanol, to cool the vessel contents to below the desired reaction temperature, preferably in ice water, and then to add a slight molar excess (preferably about 10 mol %) of neat fluoro(alkyl vinyl ether) dropwise (if a liquid, as is the case with PPVE and PEVE), or to admit the fluoro(alkyl vinyl ether) if a gas, as is the case with PMVE, into the reaction mixture that is in a pressure vessel. As the reaction proceeds, product (the adduct) separates as a second liquid phase (the product layer) at the bottom of the reaction vessel. That is, the adduct is in the heavier layer. At the end of the reaction, the product layer is separated, washed with water, optionally dried, and vacuum distilled.

If 5-norbornene-2-methanol is represented as Norb-OH, the product of the reaction (adduct) with PPVE is Norb-O—CF₂—CFH—O—C₃F₇.

The chemical names of the adducts from the above described reaction of 5-norbornene-2-methanol and the perfluoro(alkyl vinyl ethers) PPVE, PEVE, and PMVE, are as follows:

With PPVE:

5-[(1,1,2,4,4,5,5,6,6,6-decafluoro-3-oxahexoxy)methyl]-bicyclo[2.2.1]hept-2-ene
   With PEVE:
5-[(1,1,2,4,4,5,5,5-octafluoro-3-oxapentoxy)methyl]-bicyclo[2.2.1]hept-2-ene
   With PMVE:
5-[(1,1,2,4,4,4-hexafluoro-3-oxabutoxy)methyl]-bicyclo[2.2.1]hept-2-ene

Although PPVE, PEVE, and PMVE are preferred fluoro(alkyl vinyl ethers), and PPVE is more preferred, other fluoro(alkyl vinyl ethers) may be used. For example the alkyl group may have up to 10 carbon atoms, may include one or more oxygens as ether linkages, may be linear, branched, or cyclic, and may be unfluorinated, partially fluorinated, or perfluorinated. If partially fluorinated, the alkyl group preferably is otherwise hydrogen-substituted. The vinyl group of the fluoro(alkyl vinyl ether) has at least one fluorine atom on a vinyl carbon, preferably at least two fluorine atoms, more preferably at least two fluorine atoms on the terminal carbon of the vinyl group, and most preferably, the vinyl group of the fluoro(alkyl vinyl ether) has three fluorine atoms, that is, the vinyl group is most preferably perfluorinated. A generic representation of the above described fluoro(vinyl alkyl ether) is C₂FₐH_{b}—O—CₓF_{y}H_{z}Oₙ where; a=1 to 3 and b=0 to 2, a+b=3; x=1 to 10, y=0 to 2x+1, z=0 to 2x+1, y+z=2x−1 or 2x+1; and n=0 to 8 with the proviso that the oxygen is ether oxygen.

The product of the reaction of 5-norbornenyl-2-methanol with fluoro(alkyl vinyl ether) described above may also be called the adduct of 5-norbornenyl-2-methanol and fluoro(alkyl vinyl ether). A generic representation of the adduct is: R—CH₂—O—C₂FₐH_{b}—O—CₓF_{y}H_{z}Oₙ where R is bicyclo[2.2.1]hept-2-enyl and is bonded to the methylene group at the 5-position; a=1 to 3 and b=1 to 3, a+b=4; x=1 to 10, y=0 to 2x+1, z=0 to 2x+1, y+z=2x−1 or 2x+1; and n=0 to 8 with the proviso that the oxygen is ether oxygen. A preferred adduct is R—CH₂—O—C₂F₄—O—CₓF_{y}Oₙ where R is bicyclo[2.2.1]hept-2-enyl and is bonded to the methylene group at the 5-position; x=1 to 10, y=2x−1 or 2x+1; and n=0 to 8 with the proviso that the oxygen is ether oxygen.

The monomer is polymerizable to homopolymer and to a wide variety of copolymers with one or more comonomer. The word “polymerized” in connection with a specific adduct of or adducts generally of 5-norbornenyl-2-methanol and fluoro(alkyl vinyl ether) in this patent application is intended refer to both homopolymer and to copolymers with one or more comonomers made from the adducts. The polymers have a wide variety of possible end uses with certain copolymers employing adducts of 5-norbornenyl-2-methanol and fluoro(alkyl vinyl ether) being suitable for use in photoresists. Comonomers for polymerization with adducts of 5-norbornenyl-2-methanol and fluoro(alkyl vinyl ether) include comonomers which are useful for polymerization with norbornene and substituted norbornenes. Fluoromonomers such as fluoroolefins, e.g., tetrafluoroethylene (TFE), hexafluoropropylene (HFP), chlorotrifluoroethylene (CTFE), trifluoroethylene, hexafluoroisobutylene, perfluoroalkyl ethylene, fluorovinyl ethers, vinyl fluoride (VF), and vinylidene fluoride (VF2) can be used. Tetrafluoroethylene is a particularly useful comonomer. The amount of TFE in the copolymer should not be so great as to introduce crystallinity. Additional comonomers include nonfluorinated monomers such as acrylic and methacrylic esters. Known polymerization techniques for norbornene and substituted norbornenes, adapted as necessary for use with the adducts, are useful for making polymers in accordance with the invention. Suitable polymerization procedures are described for example in U.S. Pat. No. 4,751,168 and WO 2004/011509. See Example 4.

### EXAMPLES

Bicyclo(2.2.1)hept-5-ene-2-methanol (called 5-norbornene-2-methanol) is available from Aldrich Chemical Company, Milwaukee Wis. USA in 98% purity as a mixture of exo and endo isomers. The material is used without further purification.

Perfluoro(propyl vinyl ether) (PPVE) 98% minimum purity, Perfluoro(ethyl vinyl ether) (PEVE) 99% minimum purity, and PMVE 99% minimum purity are available from SynQuestLabs, Alachua Fla. USA. They are used without further purification.

Hydroxyadamantyl acrylate is available from OHKA America of Milipatas Calif. USA.

### Example 1

### Synthesis of 5-[(1,1,2,4,4,5,5,6,6,6-decafluoro-3-oxahexoxy)methyl]-bicyclo[2.2.1]hept-2-ene

A one liter round bottom flask with three necks is fitted with a Teflon® coated magnetic spin bar, thermometer, and pressure equalizing dropping funnel. Dimethyl sulfoxide (DMSO) (220 g), 5-norbornene-2-methanol (80.6 g, 0.65 mole) and aqueous potassium hydroxide solution (4.2 g of 45% solution) is added to the flask and PPVE (194.0 g, 0.729 mole) is added to the addition funnel. The flask is cooled to 10° C. in a mixture of ice and water and the PPVE is added in small portions over one and one third hours. The rate of PPVE addition is controlled so that the reaction temperature did not exceed 22° C. Additional portions of aqueous potassium hydroxide (4.2 g each) are added to the reaction mixture when added PPVE totals 63 g and again when the total PPVE added has reached 144 g. As the reaction proceeds the mixture turns a hazy amber color and later forms a second liquid phase. The denser phase weighs 267.1 g and contains, by gas chromatographic (GC) analysis, 92.4% product isomers, 5.8% unreacted PPVE, and 0.2% DMSO, all weight percent. After washing with 275 mL water there is obtained 217.1 g product. The product is vacuum distilled, coming over at 68° C. at 0.9 mm Hg (120 Pa) to yield a clear and colorless, not viscous liquid. Yield of distilled product: 64%, based on 5-norbornene-2-methanol.

The structure of the product is confirmed by NMR.

¹⁹F NMR (CDCl₃, CFCl₃ internal standard) −81.95 ppm, triplet, ³J_{F,F}=7 Hz, 3F; −85.05 ppm, AB pattern, ²J_{F,F}=146 Hz, 1F; −87.51 ppm, AB pattern, ²J_{F,F}=146 Hz, 1F; −89.39 ppm, AB pattern, ²J_{F,F}=145 Hz, ⁴J_{H,F}=7 Hz, 1F; −90.54 ppm, AB pattern, ²J_{F,F}=145 Hz, ⁴J_{H,F}=7 Hz, ⁴J_{H,F}=3 Hz; −130.31 ppm, singlet; −145.00 ppm, doublet of multiplets, ²J_{H,F}=54 Hz. ¹³C NMR (major exo isomer, CDCl₃, Trimethyl silane (TMS) internal standard) 28.98 ppm, doublet, 3 Hz; 38.47 ppm, doublet, 2 Hz; 42.62 ppm, singlet; 43.98 ppm, doublet, 2 Hz; 49.65 ppm, singlet; 68.23 ppm, doublet of doublets, 5 Hz; 98.49 ppm, doublet of triplets of triplets, 244, 42 and 4 Hz; 107.11 ppm, triplet of quartets, 268 & 38 Hz; 116.17 ppm, triplet of triplets, 286 & 33 Hz; 117.63 ppm, quartet of triplets, 286 & 33 Hz; 117.90 ppm, triplet of doublets, 266 & 28 Hz; 132.25 ppm, singlet; 138.17 ppm, singlet. ¹³C NMR (minor endo isomer, CDCl₃, TMS internal standard) 29.62 ppm, doublet, 2 Hz; 38.68 ppm, singlet; 41.96 ppm, singlet; 43.74 ppm, singlet; 45.15 ppm, singlet; 68.97 ppm, triplet, 5 Hz; 117.70 ppm, triplet of doublets, 267 & 29 Hz; 136.49 ppm, singlet; −137.36 ppm, singlet.

### Example 2

### Synthesis of 5-[(1,1,2,4,4,5,5,5-octafluoro-3-oxapentoxy)methyl]-bicyclo[2.2.1]hept-2-ene

A one liter round bottom flask with three necks is fitted with a Teflon® coated magnetic spin bar, thermometer, pressure equalizing dropping funnel. DMSO (230 g), 5-norbornene-2-methanol (80.0 g, 0.656 mole) and aqueous potassium hydroxide solution (4.5 g of 45% solution) are added to the flask and PEVE (152.0 g, 0.704 mole) is added to the addition funnel. The PEVE is added as in the example during a one hour period. An additional 4.5 g of aqueous potassium hydroxide is during the addition. The reaction mixture consisted of 272.7 g of a dark brown less dense phase and 162.6 g of a light brown more dense phase. The lower phase is twice washed with an equal volume of water to yield 114.0 g product which is vacuum distilled at 50-52° C. at 3 mm Hg (400 Pa) to yield a clear and colorless, not viscous liquid. Yield is 39% based on 5-norbornene-2-methanol.

The structure of the product is confirmed by NMR.

¹⁹F NMR (CDCl₃, CFCl₃ internal standard) −86.53 ppm, singlet; 3F; −89.16 ppm, AB pattern, ²J_{F,F}=145 & 9 Hz, 1F; −89.30 ppm, AB pattern, ²J_{F,F}=145 Hz, 1F; −90.24 ppm, AB pattern, ²J_{F,F}=146, ³J_{F,H}=29 Hz, ³J_{F,F}=8 Hz & ⁴J_{F,H}=3 Hz, 1F; −91.28 ppm, AB pattern, ²J_{F,F}=146 Hz, 1F; −144.67 ppm, multiplet.

### Example 3

### Synthesis of 5-[(1,1,2,4,4,4-hexafluoro-3-oxabutoxy)methyl]-bicyclo[2.2.1]hept-2-ene

A 600 mL evacuated autoclave is charged with DMSO (230 g), 5-norbornene-2-methanol methanol (80.4 g, 0.659 mole) and aqueous potassium hydroxide solution (9.0 g of 45% solution). PMVE (118.2 g, 0.712 moles) are added as vapor to keep a pressure of 50 psig (450 kPa). After one day the reaction mixture is transferred to a separatory funnel. The reaction mixture consisted of 393.5 g of a dark brown less dense phase and 24.3 g of a more dense phase. The combined phases are washed twice with an equal volume of water. The separation is aided by the addition of a small amount of sulfuric acid. The lower phase of 153.6 g is vacuum distilled at 38-42° C. at 1 mm Hg (130 Pa). Yield is 63% based on 5-norbornene-2-methanol.

The structure of the product is confirmed by NMR. ¹⁹F NMR (CDCl₃, CFCl₃ internal standard) −60.44 ppm, doublet, 3 Hz, 3F; −89.54 ppm, AB pattern multiplets, ²J_{F,F}=145 Hz, 1F; −90.17 ppm, AB pattern doublet of doublet of doublets, ²J_{F,F}=145 Hz, ³J_{F,H}=24 Hz, ³J_{F,F}=7 Hz & ⁴J_{F,H}=2 Hz 1F; −145.48 ppm (minor isomer), doublet of multiplets, ²J_{F,H}=53 Hz; −145.57 ppm (major isomer) doublet of multiplets, ²J_{F,H}=54

### Example 4

### Polymerization of

5-[(1,1,2,4,4,5,5,6,6,6-decafluoro-3-oxahexoxy)methyl]-bicyclo[2.2.1]hept-2-ene

5-[(1,1,2,4,4,5,5,6,6,6-decafluoro-3-oxahexoxy)methyl]-bicyclo[2.2.1]hept-2-ene is polymerized with tetrafluoroethylene (TFE) and hydroxyadamantyl acrylate (HAA) according to the general procedure disclosed in WO 2004/011509 Example 2 with 5-[(1,1,2,4,4,5,5,6,6,6-decafluoro-3-oxahexoxy)methyl]-bicyclo[2.2.1]hept-2-ene being substituted for the hexafluoroisopropanol substituted norbornene (NB—F—OH). ¹³C NMR analysis shows the resulting polymer to contain units derived from TFE, HAA, and 5-[(1,1,2,4,4,5,5,6,6,6-decafluoro-3-oxahexoxy)methyl]-bicyclo[2.2.1]hept-2-ene in the ratio of approximately 25:25:50.

What is claimed is:

1. R—CH₂—O—C₂FₐH_{b}—O—CₓF_{y}H_{z}Oₙ where R is bicyclo[2.2.1]hept-2-enyl and is bonded to the methylene group at the 5-position; a=1 to 3 and b=1 to 3, a+b=4; x=1 to 10, y=0 to 2x+1, z=0 to 2x+1, y+z=2x−1 or 2x+1; and n=0 to 8with the proviso that the oxygen is ether oxygen.

2. R—CH₂—O—C₂F₄—O—CₓF_{y}Oₙ where R is bicyclo[2.2.1]hept-2-enyl and is bonded to the methylene group at the 5-position; x=1 to 10, y=2x−1 or 2x+1; and n=0 to 8 with the proviso that the oxygen is ether oxygen.

3. 5-[(1,1,2,4,4,5,5,6,6,6-decafluoro-3-oxahexoxy)methyl]-bicyclo[2.2.1]hept-2-ene.

4. 5-[(1,1,2,4,4,5,5,5-octafluoro-3-oxapentoxy)methyl]-bicyclo[2.2.1]hept-2-ene.

5. 5-[(1,1,2,4,4,4-hexafluoro-3-oxabutoxy)methyl]-bicyclo[2.2.1]hept-2-ene.

6. Polymerized R—CH₂—O—C₂FₐH_{b}—O—CₓF_{y}H_{z}Oₙ where R is bicyclo[2.2.1]hept-2-enyl and is bonded to the methylene group at the 5-position; a=1 to 3 and b=1 to 3, a+b=4; x=1 to 10, y=0 to 2x+1, z=0 to 2x+1, y+z=2x−1 or 2x+1; and n=0 to 8 with the proviso that the oxygen is ether oxygen.

7. Polymerized 5-[(1,1,2,4,4,5,5,6,6,6-decafluoro-3-oxahexoxy)methyl]-bicyclo[2.2.1]hept-2-ene.

8. Polymerized 5-[(1,1,2,4,4,5,5,5-octafluoro-3-oxapentoxy)methyl]-bicyclo[2.2.1]hept-2-ene.

9. Polymerized 5-[(1,1,2,4,4,4-hexafluoro-3-oxabutoxy)methyl]-bicyclo[2.2.1]hept-2-ene.
